(19) United States
(12) Patent Application Publication
(10) Pub. No.: US 2012/0143346 A1
(43) Pub. Date: Jun. 7, 2012

(54) MODULAR HIP STEM SYSTEM

(75) Inventors: Tracy Rickels, Hewitt, NJ (US); William Huffnagle, Franklin Lakes, NJ (US)

(73) Assignee: HOWMEDICA OSTEONICS CORP., Mahwah, NJ (US)

(21) Appl. No.: 12/957,814

(22) Filed: Dec. 1, 2010

(51) Int. Cl. A61F 2/36

(52) U.S. Cl. 623/23.15

(57) ABSTRACT

A kit of modular prosthetic hip components includes a first neck having a coupling element thereon. First and second hip stems each have a coupling element at a proximal end thereof for connecting to the coupling element of the first neck. The first and second hip stems have a progressively larger medial-lateral dimension in a proximal portion thereof. The coupling element on each of the first and second hip stems is located at a different medial-lateral location thereon with the coupling element located more medially and proximally on the second stem than the first stem. The first neck forms an identical first neck angle with a proximal-distal axis of the respective first and second stems. The coupling elements on the first and second stems locate a head mounted on the first neck in the same proximal-distal location but a different medial-lateral location with respect to an acetabulum.

### BACKGROUND OF THE INVENTION

This invention relates generally to a system of modular femoral components for a hip joint prosthesis and, more particularly, is directed to the shape of the intramedullary stem which is inserted into the surgically prepared medullary canal of the proximal femur for fixation or anchorage of the prosthesis into the surrounding supportive cortical bone and also relates to the location for insertion of a neck element into the stem.

As is well-known in the art, the intramedullary stem or blade-like portion of the femoral component of a hip joint prosthesis is inserted into the prepared medullary canal of the proximal femur for fixation within the surrounding bone structure. This fixation occurs by either bone cementing which employs methyl methacrylate, or by biological fixation employing a mechanism of bone ingrowth within minute voids or porosities of a specially applied porous-like metal alloy surface structure. This porous surface structure is metallurgically integrated onto the surface of the prosthesis stem at specially selected locations.

With this in mind, a congruent fit between the femoral prosthesis stem and prepared proximal medullary canal of the femur is an important technical and clinical consideration. One factor that affects the ability to achieve a congruent fit is the natural shape of the proximal medullary canal prior to the canal being prepared to receive the prosthesis stem. It is generally recognized that there are two shape extremes of the natural proximal femoral medullary canals. The first shape extreme is often referred to as a “stove pipe” shape, and the second type of shape is often referred to as a “champagne flute” (conical) or more narrow distally) shape. The third basic shape is the most usual and falls between “champagne” and “stove pipe”. It would be advantageous for a surgeon to have available femoral prosthesis stems that are suited for a particular type of proximal femoral medullary canal. Advantages lie in ease of preparation of the medullary canal and the potential for a more congruent fit between the stem and canal.

Additionally, besides attempting to achieve a stable mechanical press-fit within the proximal femoral canal for porous ingrowth fixation (and for cementation, as well), the surgeon must attempt to position the center of the bearing head of the femoral prosthesis coincident with the articular center of the hip joint for duplication of the natural functional biomechanical characteristics of the hip. The two principal spacial components of femoral head position include proximal-distal offset distance (proximal-distal elevation from the collar or neck resection level to the femoral head center) for leg length considerations and medial-lateral (M-L) offset (medial-lateral dimension between the medullary stem center-line and the head center) for attainment of proper hip joint function and power characteristics.

One way the femoral head position can be changed by the surgeon is by selecting a neck from a range of different length modular neck components. Also head components which allow discrete incremental increases in both the proximal-distal (P-D) and medial-lateral offsets in relation to a neck can be provided. The neck angles of the modular necks can also be varied to change P-D and M-L offsets. Variation in just a medial-lateral (M-L) component of the femoral head position is achieved by selecting an alternative femoral component design, which incorporates a larger (M-L) offset. Such a femoral component may not be readily available for a given distal stem size within the same prosthesis system, and accordingly, may involve consideration of a long lead-time custom device, or more probably, consideration of another prosthesis system may have to be explored.

Another possible way the desired proximal-distal femoral head offset can be achieved is by preparing the proximal femoral canal to accept the prosthesis at a higher position or by selecting an alternative femoral component design of proper distal diameter, but with a broader proximal-transverse or a proximal M-L intramedullary stem width. This allows the prosthesis to seat at a higher position within the femur. The former technique for seating the femoral component at a higher position can result in larger stem/bone interface gaps proximal-medially, a condition reflective of a less desirable, and possibly less stable, stem fit. Conversely, to lower the vertical head offset position, a femoral prosthesis stem of narrower proximal-transverse width can be employed, which allows the prosthesis to seat at a lower position within the femur. Lower prosthesis seating may also result in an inexact fit between the stem and bone, especially if the femur was surgically prepared for a larger proximal stem design of different proximal-medial curvature. Again, these adjustments in femoral head position must be surgically achieved while coincidentally attaining a tight, stable intramedullary stem fit within the proximal femur with minimal interface gaps between the stem and bone.

Another way to provide a surgeon with an ability to alter the position of the center of the prosthetic femoral head relative to the prosthetic femoral stem axis is to provide monolithic stems that, for a given size, offer either different neck angles or pure P-D and/or M-L offsets. In order to determine which type of angle offset is most appropriate, some prosthetic implant systems offer the ability to attach trial necks to the tools used to shape the proximal medullary canal of the femur. This assembly permits a trial reduction of the joint that will reveal if the choice of implants is appropriate for the patient. However, a disadvantage to these systems is that they do not allow a surgeon to trial off of the actual stem implant. Variations in position between the proximal medullary femoral canal shaping tools during a trial reduction and the actual position of the stem implant can result in the femoral head implant center being positioned in a slightly different place than intended relative to the femoral prosthesis stem axis and neck resection level.

Another way to provide a surgeon with an ability to alter the position of the center of the prosthetic femoral head relative to the prosthetic femoral stem axis is to provide a femoral prosthesis stem that is able to accept any one of a number of modular femoral neck prosthesis options. These femoral neck prosthesis options can be configured to allow any practical number of neck angles and lengths or independent P-D and M-L offsets. The disadvantages to these modular stem-neck implant systems is that due to the junction between the two mating parts the resulting implant may not be as strong as a monolithic implant of the same geometry. However, the advantage is that the femoral head center may be able to be more accurately placed relative to the femoral prosthesis stem axis and neck resection level.

In the past, stems with longer monolithic and or modular necks had to be used to achieve the extra offset that patients on the high end of the spectrum for neck length and head offset needed. Alternately one can change the neck angle such as 127° or 132° for a given size stem. This results in two different head center locations

Neck designs in both monolithic and modular stem designs are subjected to a very high amount of stress when loaded in the hip. The longer the neck is for a given patient's body weight, the more stress is applied to both the neck and the mating coupling feature on stem. Current modular neck designs have been developed with relatively expensive materials that are much stronger in order to deal with this high stress. In some cases, modular neck length is also limited for a given set of stem sizes due to the maximum stress limits of the materials that are currently used. This results in a lack of appropriate head offset values for some patient's anatomy. Again the neck angle can be changed with different modular necks. Thus the neck angle can be changed independently.

### BRIEF SUMMARY OF THE INVENTION

The key difference for the system of the present invention is that a single stem size can have variations in the position of the mating feature for the modular neck and this leads to the ability to give the patient more head medial-lateral offset without changing the neck length, neck angle and/or proximal-distal head position. An alternate embodiment shifts only the P-D head location without changing the M-L location.

Each stem size has two or more variations in medial curvature for a given size. The anterior/posterior widths of the stem could also be varied along with the medial calcar offset values and head offset values relative to the proximal axis of the femur. The first variation is designed to fit into patients with lower than average sets of medial calcar head offsets. The second stem variation will be designed to fit into patients who have a higher than average set of medial calcar offset values and corresponding medial-lateral head offset values relative to the proximal axis of the femur. A third variation may fit into patients who have average medial calcar offset values and head offset values.

In the modular neck stem design, the location of the neck coupling element which is typically a mating female pocket that would receive the modular neck in the first variation of the modular stem design will be shifted towards M-L the low end of the distribution of data for medial-lateral head offset. The location of the female pocket or tapered bore in the second variation of the modular stem design will be shifted towards the high end of the population distribution of data for M-L head offset. The location of the female pocket or tapered bore in the third variation of the modular stem design would be positioned near the average values for both medial calcar offset and M-L head offset. In addition a second M-L offset of the pocket in which the neck is inserted will give an identical M-L head offset. Thus a shorter neck can be used to obtain the desired M-L offset.

Statistical analysis data from a virtual bone database has been utilized to determine the appropriate medial curvatures and corresponding head offsets for each variation. Just like in the conventional modular neck designs, the medial-lateral head offset can be controlled both by the modular neck angle to the longitudinal stem axis, neck length and the position of the receiving pocket or tapered bore that is located in the proximal stem. The key difference for this system is that a single stem size can have variations in medial-lateral head offset without changing the neck length, neck angle, and/or head proximal-distal position. The standard deviation in data for M-L head offset in a large group of patients on average is approximately +/−2.5 mm and approximately 2-3 mm over the entire range of bone sizes analyzed. This range could be increased or decreased if additional optimization is required. The subset of patients shown in FIG. 4 was selected based on a predicted fit between the M-L canal width at 20 mm below the lesser trochanter for the most common stem size that is being developed for the modular stem system of the present invention and the canal measurements for same location in each of the patient's femurs. If more variations of modular stems were to be designed for each stem size (i.e. 3 or more variations), then a 5 mm M-L offset difference shown in FIG. 6 could either be maintained, reduced to a lower value, or increased to cover a larger distribution of patients or any combination of these three options could be combined.

The modular stem/neck design with two or more mating pocket positions for each stem size allows a surgeon to reconstruct leg length and medial-lateral head offset without adding extra neck length in patients who have a higher than average head position and corresponding medial canal offset values along the length of the calcar. This results in a stronger system with a shorter moment arm.

Each stem size has multiple medial curvature options. One version of this stem design has a modular neck pocket and would utilize modular necks in a new way that will allow surgeons to attain the appropriate medial-lateral head offset range, leg length range and anteversion angle range with reduced neck lengths for patients that require high head offsets and longer then normal neck lengths.

A kit of modular prosthetic hip components is provided which includes a first neck element having a coupling element thereon. The kit has first and second hip stem elements each having a coupling element at a proximal end thereof for connecting to the coupling element of the first neck element. The first and second stem elements each have a progressively larger medial-lateral dimension in a proximal portion thereof. The coupling element or pocket on each of the first and second stem elements is located at a different medial-lateral location thereon. The coupling element or pocket is located more medially and proximally on the second stem element than the first stem element. The first neck element forms an identical first neck angle with a proximal-distal axis of the respective first and second stem elements and locates a femoral head mounted on the neck element in the same proximal-distal location but a different medial-lateral location with respect to the patient's acetabulum. By not increasing the neck length the moment arm between the loading point of the femoral head and the coupling element or pocket on the stem is not increased.

The dimension in the medial-lateral direction at a proximal most surface of each stem element increases in increments from the first stem element to the second stem element such as in increments of 2.5 mm. The distance between the proximal-distal axis of each stem and a lateral side of the first and second stem elements are equal. The first neck element may have a second coupling element, such as a male taper for attaching a prosthetic femoral head. The kit may further comprise a second neck element forming a second angle with the proximal-distal axis of a respective first, second and third stem elements. The second neck angle is different than the first neck angle such as 135° and 145°. The first and second stem elements each have an identical distal stem portion including a distal tip of each stem element. The first neck element has a longitudinal axis wherein a second end of the first neck element along the longitudinal axis when the neck and stem coupling elements are assembled is offset in the medial direction a distance which increases in increments from the first stem element to the second stem element. The coupling element on each stem element may be a tapered bore which has a central axis and the central axis moves medially in the increments (2.5 mm) from the first stem element. Each stem element has a superiorly facing proximal surface including the tapered bore, the superiorly facing proximal surface moving superiorly in incremental distances from the first stem element to the second stem element. The first stem element has a coupling element such as the tapered bore located more laterally and distally than the second stem element. A medial side of the first and second stem elements may be curved with the curve of the second stem element having a smaller radius than the first stem element. Generally the curve starts distally approximately 40 mm below a proximal most medial edge of the proximal portion of the stem element.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

The kit or system may also have groups of different size stem elements (A-P dimension, length and M-L dimensions), each group having at least two stems with identical distal portions which distal portions differ in dimension from group to group.

### BRIEF DESCRIPTION OF THE DRAWINGS

FIG. 1a shows a prior art femoral component, including a modular spherical head with the neck extending at an angle of 127° with respect to the longitudinal axis of the stem;

FIG. 1b is a prior art femoral component similar to that of FIG. 1a with the exception that the neck angle is 132°;

FIG. 2a is a prior art head for a femoral component having a pocket for receiving the proximal end of a neck component located in a first location with the part spherical head;

FIG. 2b is a part spherical head for a femoral component having the same diameter as that of FIG. 2a, but including a skirt, which moves the location of the pocket in the head for receiving the proximal end of the neck component in a distal direction;

FIG. 3a is a prior art femoral component, including a modular neck having a first length connecting a part spherical head to the stem component;

FIG. 3b is a second prior art femoral component somewhere to that of FIG. 3a with a longer neck component;

FIG. 4 shows a compilation of head offsets versus medial offsets at the calcar from various virtual bone databases obtained from various studies of actual human femurs;

FIG. 5 is an enlarged view of a typical proximal femur showing the medial offset from the anatomic axis of the proximal femur which varies according to the medial offset at the bottom of FIG. 4; and

FIG. 6 is an overlay of two femoral components of the present invention differing in the medial and proximal dimensions while placing a head at a 5 mm medial-lateral offset but at the same proximal-distal location with regard to an acetabulum of a patient.

FIG. 7 shows part of a kit of components, including two identically sized stems having a pocket shifted in a medial-lateral direction but having the same proximal-distal location; and

FIG. 8 shows a first and second modular neck for use with the femoral stems of FIG. 7 with the two necks having a different length.

### DETAILED DESCRIPTION

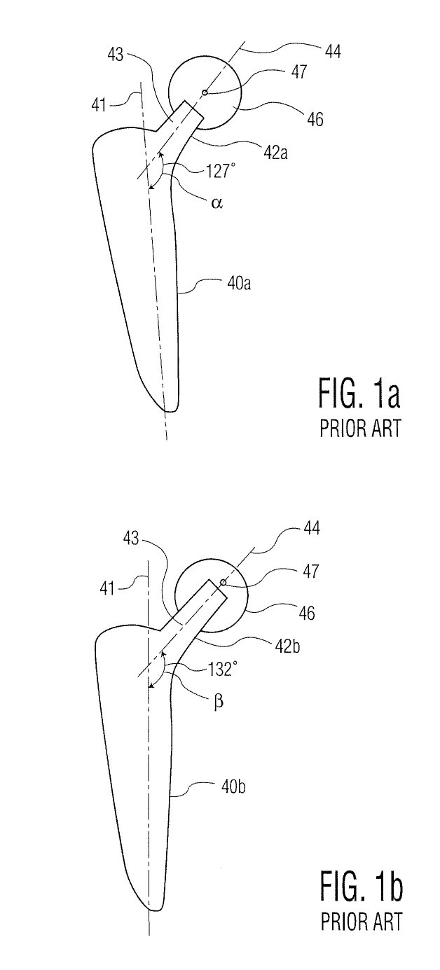

Referring to FIGS. 1a and b there is shown a first stem component 40a and a second stem component 40b. Stems 40a, 40b include a longitudinal axis 41, which extends in a generally proximal-distal direction. Stems 40a and 40b include necks 42a and 42b having a trunion 43 at the proximal most end thereof. Necks 42a, 42b extent along an axis 44. In the stem of FIG. 1a, the angle between axis 44 and axis 41 is proximally a 127°, whereas in FIG. 1b it is 132°. A part-spherical modular head 46 is utilized on both necks 42a and 42b. Heads 46 has a center 47. Because the neck angle alpha (127°) and beta (132°) are different, the location of head center 47 changes both in a proximal-distal direction and in a medial-lateral direction. This is one way that the prior art has addressed changing the center of rotation of prosthetic femoral component with respect to a prosthetic acetabular component.

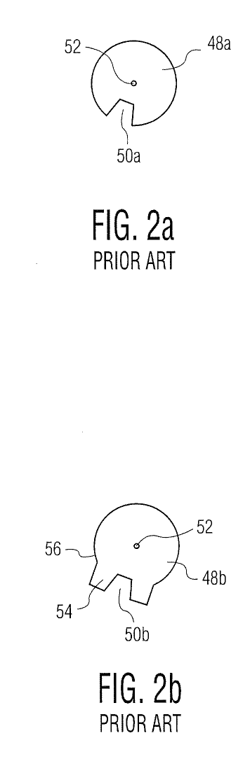

A second way of changing the center location is shown in FIGS. 2a and 2b, which show two heads 48a and 48b having the same spherical radius but having a pocket 50a and 50b located in different locations with respect to a center 52 of each head 48a, 48b. This is accomplished by adding a skirt 54, which extends distally from a bottom surface 56 of head 48b so that pocket 50b is located along the femoral component neck axis more distally than head 48a. Again, this changes the medial-lateral and proximal-distal location of the head center 52.

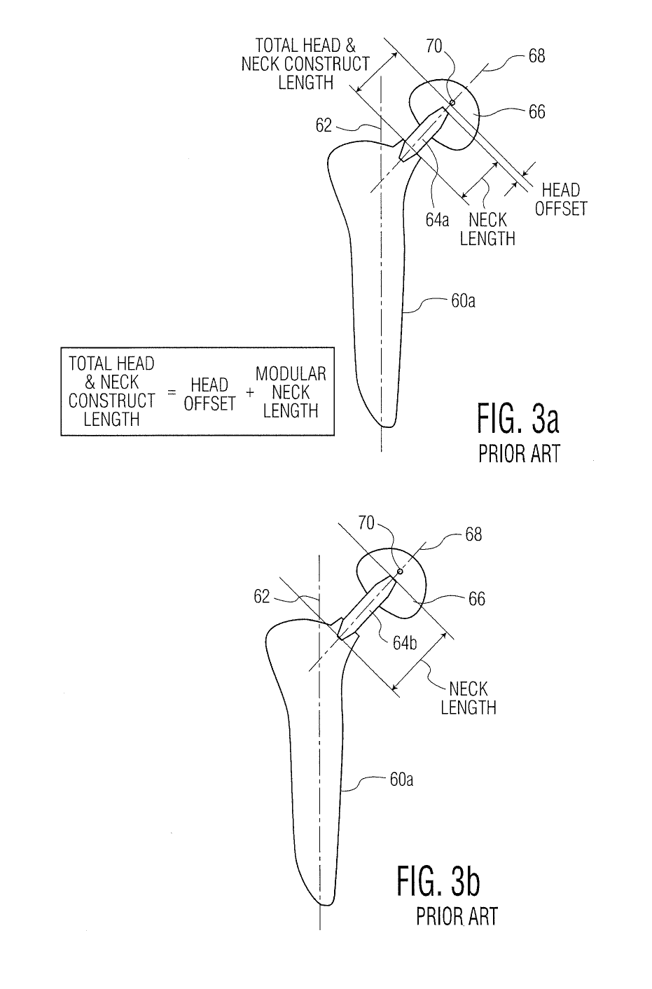

Yet another way of the prior art changes the location of the center of the spherical head with respect to the acetabulum is shown in FIGS. 3a and 3b, which include distal femoral components 60a. Femoral components 60a include a central axis 62 and a modular neck 64a and 64b of different lengths connecting the identical components 60a with part-spherical head 66. Necks 64a and 64b lie along an axis 68, which forms an identical angle with axis 62 of both stems 60a and 60b. Again, the head center 70 location is changed in both the medial-lateral and proximal-distal directions on switching from modular neck 64a to 64b.

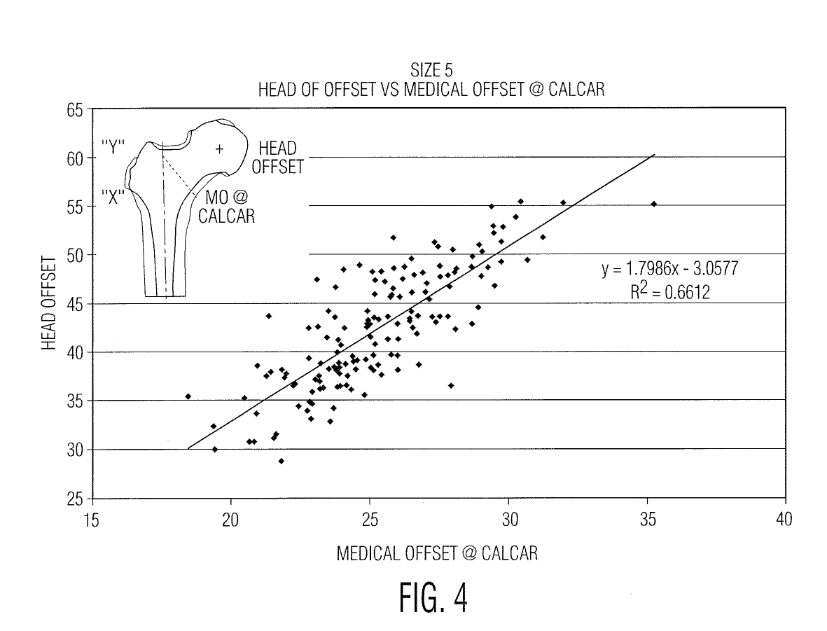

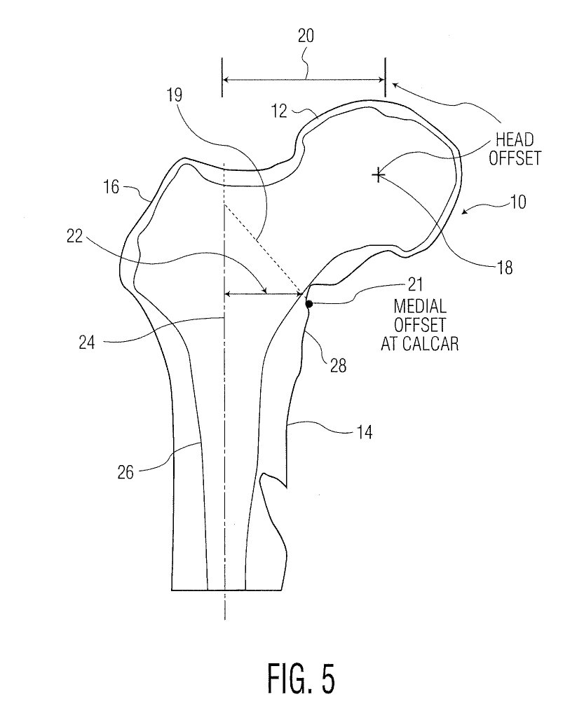

Referring to FIG. 4 there is shown a compilation of data points obtained from various studies of the proximal femur showing the medial-lateral head offset versus medial offsets at the calcar in millimeters. As shown, a majority of the population assessed has femoral head medial-lateral offsets of 35 to 50 mm and corresponding medial offsets at the calcar between 23 and 30 millimeters. Referring to FIG. 5, there is shown a typical proximal femur 10 having a head portion 12, a medial side 14, a greater trochanteric area 16 and a femoral head center 18. Line 19 represents the proposed proximal femoral resection level to point 21 on the calcar. As shown in FIG. 5, the medial-lateral head offset is designated as distance 20 and the medial calcar offset is designated as the distance along line 22. An axis 24 extends through an intramedullary canal of the femur 26. Axis 24 represents an anatomic axis of the proximal femur. The line 22 represents the medial offset extending from the calcar area 28 and perpendicularly intersects axis 24.

The size of the femoral component in the medial dimension, are selected to provide the largest coverage of individuals shown in FIG. 4.

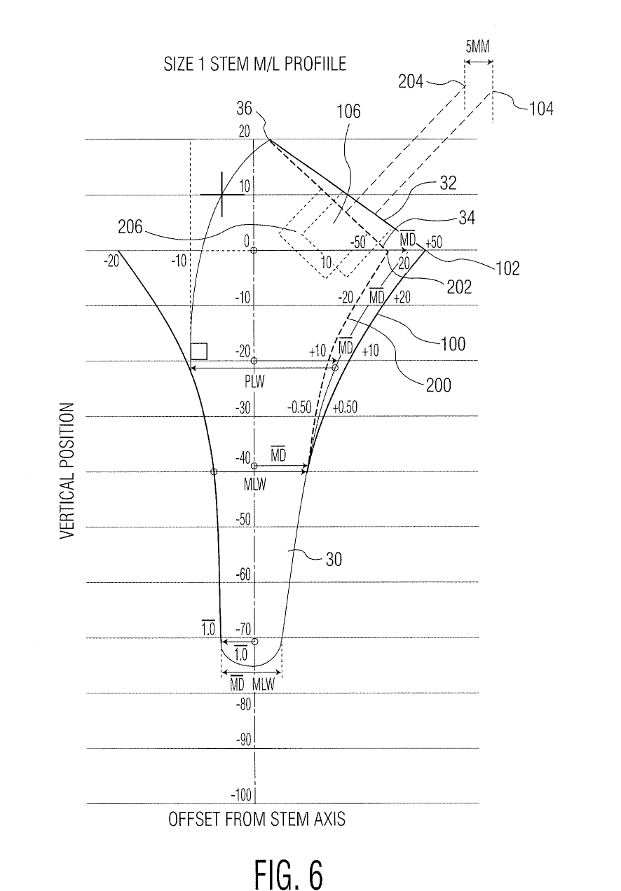

Referring to FIG. 6 there is shown a first femoral component 100 and a second femoral component 200. In all cases, femoral components 100 and 200 have an identical distal tip 30. Typically the identical distal portion 30 starts about 50 millimeters below the most medial-proximal point on the femoral component which is designated at points 102 and 202 of FIG. 6. Both points 102 and 202 are at the same proximal distal level with respect to the acetabulum of a particular patient at implantation. Each femoral component 100, 200 has a corresponding female tapered cavity or bore 106 and 206 respectively. Such tapered cavities are well known in modular femoral components and each cavity 106, 206 is identical with the exception that moving from stem 100 to stem 200, the cavity is shifted both in the medial-lateral direction and the proximal-distal direction. Consequently an identical neck portion placed in cavity 106, 206 would locate a head center at points 104, 204 respectively. The head center would be in the same proximal-distal location with respect to an acetabulum of a patient in which stem 100 or 200 is implanted. However, the head would be shifted in the medial-lateral direction, preferably 5 mm, as discussed below. Thus, a kit of parts including stems 100, 200 can be supplied with various angle and length neck elements (need to show) having a coupling element such as a nail conical trunion for insertion into coupling elements 106, 206 can be provided which, when a neck element of a given length or angle is assembled to either stems 100, 200 produces a head location which is the same in the proximal-distal direction however is offset in the medial-lateral direction.

As discussed above, stem 100 defers from stem 200 in that the calcar area is shifted medially and proximally on moving from stem 200 to stem 100. The medial shift is approximately the same as the shift in the locations of cavity 206 to cavity 106. Thus, when moving from point 202 to point 102, a shift may typically be 5 mm which is similar to the M-L head offset difference between point 204 and point 104. The proximal face 32 and 34 of stems 100 and 200 are rotated superiorly from a point 36 on the lateral side of the femoral component 100, 200. The distance of rotation from location 34 on stem 200 to location 32 on stem 100 is sufficient to move cavity 106 superiorly with respect to cavity 206 a sufficient distance to place head centers 104, 204 in the same proximal distal location with respect to a specific patient's acetabulum. Thus all stem dimensions lateral of center line 24 stay the same.

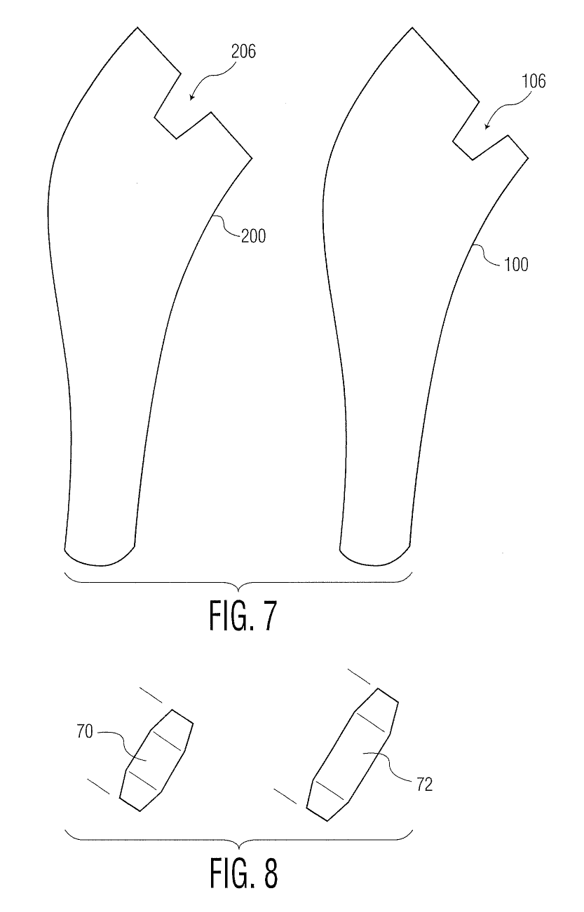

Referring to FIGS. 7 and 8 there is shown a kit of stems and modular necks, including the stems 100 and 200 shown in FIG. 6. Pockets 106 and 206 are shifted in a medial-lateral direction, but the proximal most stem surface is raised in stem 106 so that the proximal-distal location of a head center for any given neck size is in the same location. Thus a part-spherical head such as is shown in FIG. 1a can be utilized with neck 70 or 72 of FIG. 8 in a well-known manner so that such the head mounted on neck 70, when moved from pocket 106 to pocket 206, moves the head center 104, 204 5 mm in the medial-lateral direction as shown in FIG. 6. If it is desired to change in the proximal-distal location of the head center, then a longer neck such as 72 can be utilized in either pocket 106 or 206, which again results in the same medial-lateral offset albeit with a different proximal-distal offset than that produced by neck 70. Of course, three or more stems having different offset pockets and three or more necks can be utilized in the kit to produce a larger number of medial-lateral offsets with the necks of different lengths producing a different proximal-distal offset.

As could be understood by one skilled in the art, the pocket could also be shifted in the anterior-posterior direction and the proximal-distal direction.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A kit of modular prosthetic hip components comprising:
a first neck element having a coupling element thereon;
a first and second hip stem element each having a coupling element at a proximal end thereof for connecting to the coupling element of the first neck element, the first and second hip stem elements each having an identical size distal portion with a distal tip followed proximally by a divergent transition portion and the second hip stem element has a larger medial-lateral dimension at the same proximal location than the first hip stem element;
the coupling element on each of the first and second hip stem elements located at a different medial-lateral location thereon with the coupling element located more medially and proximally on the second hip stem element than the first hip stem element;
the first neck element forming the same neck angle with a central proximal-distal axis of the distal stem portion of the respective first and second hip stem elements and the first neck element when mounted on the first and second stem elements locating a prosthetic femoral head mounted on the neck element in the same proximal-distal location but a different medial-lateral location with respect to an acetabulum.

2. The kit as set forth in claim 1 wherein a dimension in the medial-lateral direction at a proximal most surface of each stem element increases medially in increments from the first stem element to the second stem element.

3. The kit as set forth in claim 2 wherein the increments are equal.

4. The kit as set forth in claim 2 wherein the distance between the proximal-distal axis and a lateral side of the first and second stem elements are equal.

5. The kit as set forth in claim 1 wherein the distance between the proximal-distal axis and a lateral side of the first and second hip stem elements are equal.

6. The kit as set forth in claim 1 wherein the first neck element has a second coupling element for attaching the prosthetic femoral head.

7. The kit as set forth in claim 1 further comprising a second neck element forming a second angle with the proximal-distal axis of the respective first and second hip stem elements, the second neck angle being different than the first neck angle.

8. The kit as set forth in claim 1 wherein the first neck element has a longitudinal axis, wherein a second end of the first neck element along the longitudinal axis, when the neck and first and second hip stem coupling elements are assembled is offset in the medial direction a distance which increases in increments from the first stem element to the second stem element.

9. The kit as set forth in claim 8 wherein the increment is 2.5 mm.

10. The kit as set forth in claim 8 wherein the coupling element on each stem element is a tapered bore which has a central axis and the central axis moves medially with respect to the proximal-distal axis in the increments from the first hip stem element to the second stem element.

11. The kit as set forth in claim 10 wherein each stem element has a superiorly facing proximal surface including the tapered bore, the superiorly facing proximal surface moving superiorly in incremental distances from the first stem element to the second stem element.

12. The kit as set forth in claim 1 wherein the kit further comprises a third hip stem element having a coupling element located more medially and proximally than the second stem element.

13. The kit as set forth in claim 12 wherein the third hip stem element has an identical distal portion of the first and second hip stem element.

14. The kit as set forth in claim 1 wherein a medial side of the first and second stem elements is curved with the curve of the second stem element having a smaller radius than the first stem element.

15. The kit as set forth in claim 14 wherein the curve starts distally approximately 40 mm below a proximal most medial edge of the proximal most portion of the stem element.

16. The kit as set forth in claim 11 wherein the anterior-posterior dimension at the superiorly facing proximal surface is greater in the second hip stem element than the first hip stem element.

17. The kit as set forth in claim 1 further comprising a second group of at least two hip stem elements all having identical distal portions which distal portions have a different dimension than the identical distal portions of the first and second hip stem elements.
